(19) United States
(12) Patent Application Publication
(10) Pub. No.: US 2005/0265997 A1
(43) Pub. Date: Dec. 1, 2005

(54) Cancer treatment method by inhibiting MAGE gene expression or function

(75) Inventors: B. Jack Longley JR., Madison, WI (US); Bing Yang, Madison, WI (US)

Correspondence Address: CROWELL & MORING LLP;INTELLECTUAL PROPERTY GROUP, P.O. BOX 14300, WASHINGTON, DC, 20044-4300, US

(73) Assignee: WISCONSIN ALUMNI RESEARCH FOUNDATION, Madison, WI (US)

(21) Appl. No.: 11/137,415

(22) Filed: May 26, 2005

Related U.S. Application Data
(60) Provisional application No. 60/574,224, filed on May 26, 2004.

(51) Int. Cl. A61K 39/395; A61K 48/00

(52) U.S. Cl. 424/141.1; 514/44

(57) ABSTRACT

Method for inhibiting tumor cell formation or tumor cell growth, and method for inducing apoptosis in sperms, the method comprising administering to a patient in need thereof an antagonist that inhibits MAGE gene expression or MAGE protein function. Preferably, the antagonist is an anti-MAGE antibody, an antisense molecule, an siRNA molecule, a molecule for forming a triplex nucleic acid molecule with a MAGE encoding polynucleotide, or a small molecule inhibitor of MAGE function. Also disclosed are pharmaceutical compositions comprising the same, and method for screening a substance that inhibits MAGE gene expression of MAGE protein function.

### CROSS REFERENCE TO RELATED APPLICATIONS

This application claims the priority of U.S. Provisional Application No. 60/574,224, filed May 26, 2004, the disclosure of which is expressly incorporated by reference herein.

### FEDERAL GOVERNMENT INTEREST

This invention was made with United States government support under a grant from the National Institutes of Health (NIH), Grant Number NIH AR043356. The United States has certain rights to this invention.

### BACKGROUND OF THE INVENTION

The first member of the melanoma associated antigen gene (MAGE) family was identified in melanomas (van der Bruggen et al., 1991, A gene encoding an antigen recognized by cytolytic T lymphocytes on a human melanoma. Science, 254:1643-7). Subsequently, many new members of the family have been identified and currently the MAGE gene family comprises dozens of related genes divided into various clusters, for example MAGE-A, B, C, D, E, F, G, H, L, dMAGE and aMAGE (De Plaen et al., 1994, Structure, chromosomal localization and expression of twelve genes of the MAGE family. Immunogenetics, 40:360-9. Lurquin et al. 1997. Two members of the human MAGEB gene family located in Xp.21.3 are expressed in tumors of various histological origins. Genomics, 46:397-408. Lucas et al., 1998, Identification of a new MAGE gene with tumor-specific expression by representational difference analysis. Cancer Res., 58:743-52. Chomez et al., 2001, An overview of the MAGE gene family with the identification of all human members of the family. Cancer Res., 61:5544-51).

These MAGE genes are expressed in many human tumors of different histological types, but are silent in normal cells with the exception of testis (see e.g. Jungbluth et al., 2000, Intl. J. Cancer 85:460-465, Yakirevich et al., 2003, Applied Immunohistochemistry & Molecular Morphology 11:37-44, and Gaskell et al., 2004, Biol. Reproduction 71:2012-2021). Some of them are also expressed in neural tissues or placenta. Male germ line cells and placenta do not express MHC class I molecules and are therefore incapable of presenting antigens to CTL (Haas et al., 1988, Distribution of human leukocyte antigen-ABC and -D/DR antigens in the unfixed human testis. Am. J. Reprod. Immunol. Microbiol. 18: 47-51; Hunt et al., 1992, Characterization of peptides bound to the class I MHC molecule HLA-A2.1 by mass spectrometry. Science 255: 1261-3).

MAGE family proteins are known to be expressed in many melanomas and T-cell and B-cell lymphomas and leukemia, and the present inventors have recently detected expression of MAGE family members in malignant mast cells.

Because MAGE family proteins are expressed in many tumors but have very limited expression in normal cells, they are attractive as specific markers for cancer diagnosis, and also represent a potential point of attack against malignant tumors that would allow therapies to target specifically the tumors without harming normal cells. Several strategies have been developed, including stimulation of immune responses to MAGE proteins, with the expectation that a specific anti-tumor effect may result. Clinical trials involving defined tumor-specific shared antigens have been and are being performed in melanoma patients, and tumor regressions have been observed in a minority of patients (Marchand et al., 2001, Biological and clinical developments in melanoma vaccines. Exp Opin. Biol. Ther., 1:497-510. Jger et al., 2002, Clinical cancer vaccine trials. Curr. Opin. Immunol., 14:178-82. Marchand et al., 1999, Tumor regressions observed in patients with metastatic melanoma treated with an antigenic peptide encoded by gene MAGE-3 and presented by HLA-A1. Int. J. Cancer, 80:219-30. Thumer et al., 1999, Vaccination with MAGE-3A1 peptide-pulsed mature, monocyte-derived dendritic cells expands specific cytotoxic T cells and induces regression of some metastases in advanced stage 1V melanoma. J. Exp. Med., 190:1669-78). However, these immunologic therapies require an intact and functioning immune system in the patient and have met only with limited success.

It now appears that the MAGE genes are frequently expressed in tumor cells because they appear to be prone to demethylation and thus are relatively easily disregulated (Honda et al., 2004, Demethylation of MAGE promoters during gastric cancer progression. Br. J. Cancer, 90:838-43).

The function of most MAGE family members remains unknown. There is some indication that certain MAGE genes induce apoptosis, or programmed cell death. For example, MAGE-D1 (also called NRAGE) appears to promote the death of some nerve cells through apoptosis (Amir et al., 2000, NRAGE, a novel MAGE protein, interacts with the p75 neurotrophin receptor and facilitates nerve growth factor-dependent apoptosis. Neuron 27:279-288. Salehi et al., 2002, NRAGE, a p75 neurotrophin receptor-interacting protein, induces caspase activation and cell death through a JNK-dependent mitochondrial pathway. J. Biol. Chem., 277:48043-50). These studies appear to indicate that an increased expression of MAGE genes is desirable for inducing cancer cell apoptosis.

Surprisingly and contrary to the prevailing knowledge in the art, the present inventors discovered that inhibition of MAGE gene expression or MAGE protein function interferes with the growth and survival of cancer cells.

### BRIEF DESCRIPTION OF THE DRAWINGS

FIG. 1 is a Western blot showing down-regulation of MAGE-A protein during induction of apoptosis in HMC-1.1 cells at 12 and 24 hours after the administration of STI 571.

FIGS. 2a and 2b shows that both MAGE-D1 and MAGE-A siRNAs significantly inhibited growth of HMC-1.1 malignant mast cell subclone at 50 nM and 100 nM. MAGE-E1 siRNA showed significant inhibition of HMC-1.1 cell growth at 50 nM, and MAGE-A and MAGE-E1 significantly inhibited growth of the HMC-1.2 malignant mast cell subclone growth at 100 nM.

For FIGS. 2-5, the cells number was counted on the 3rd day after siRNAs transfection. Starting cell number is 100000/well (dashed lines). P<0.05 was considered as statistical significance, shown as *.

FIG. 3 shows that growth of the human melanoma line Hs-294T was inhibited by MAGE-A specific siRNA at 50 nM and by MAGE-D1 specific siRNA at 100 nM.

FIG. 4 shows another human melanoma line, A-375, was inhibited by MAGE-A specific siRNA 100 nM and MAGE-E1 specific siRNA at 150 nM.

FIG. 5 shows that none of the siRNAs (both nonspecific and specific siRNAs), showed adverse affects on a human epithelial cancer cell line, HaCaT, which does not express these MAGE proteins, confirming the specificity of the effect by showing that neither the transfection process nor the non-specific siRNAs kill the cells at the siRNA concentrations used.

FIG. 6 shows western blots of HMC-1.1 cells transfected with siRNA, indicating that the specific siRNA reagents effectively prevented the synthesis of the corresponding MAGE proteins but that non-specific siRNA reagents did not effect MAGE protein synthesis.

FIG. 7a shows a Kaplan-Meier plot comparing mice injected with melanoma cells transfected with MAGE siRNA to mice injected with melanoma cells treated with non-specific siRNA and to mice injected with untreated (control) melanoma cells. FIG. 7b depicts a comparison by Log-Rank analysis between MAGE siRNA treated melanoma growth in mice and control and nonspecific siRNA treated melanomas, and shows that melanoma growth was significantly (p<0.01) slower with MAGE siRNA treatment when compared to both control and non-specific siRNA treatments. FIG. 7c is a plot of daily tumor size, and FIG. 7d is the Linear Regression analysis of the same data, showing that tumors grew an average of 0.32 mm per day post-inoculation in the control group, 0.38 mm per day in the nonspecific group, and only 0.15 mm per day for the MAGE group. The observed differences for MAGE mice as compared with either control or nonspecific mice were statistically significant with p<0.01.

FIG. 8 shows the data, similar to FIG. 7, with treatment by intraperitoneal siRNA injections after tumor inoculation. Note that systemic treatment with MAGE siRNA effectively slowed the growth of established tumors compared to treatment with non-specific siRNA.

### SUMMARY OF THE INVENTION

The present invention provides a method for inhibiting the growth or proliferation, or inducing apoptosis, of a cell that expresses a MAGE gene, the method comprising inhibiting the expression of the MAGE gene or inhibiting the function of a polypeptide encoded by the MAGE gene in the cell.

In one embodiment, the method comprises administering to the cell a polynucleic acid molecule that specifically inhibits the expression of the MAGE gene in the cell. Preferably, the polynucleic acid molecule is a short interfering RNA molecule, or an antisense nucleic acid molecule that is specific against the MAGE gene, or a nucleic acid molecule that forms a triplex with the MAGE gene, thereby inhibiting the expression of the MAGE gene.

In another embodiment, the method comprises administering to the cell a polypeptide that specifically inhibits the function of a protein encoded by the MAGE gene. Preferably, the polypeptide is an antibody against the MAGE gene or MAGE gene product.

Suitable MAGE genes for the present invention may be a Type I MAGE gene, such a MAGE-A, MAGE-B, or MAGE-C, specifically, MAGE-A1, A3, A5, A6, A8, A9, A10, A11 or A12, or MAGE-B1, B2, B3 or B4. The MAGE gene may also be a Type II MACE gene, such as Necdin, MAGE-D, MAGE-E (E1), MAGE-F, MAGE-G, or MAGE-H.

In preferred embodiments, the method inhibits MAGE gene function in a cell A which is a cancerous or malignant or neoplastic cell. Preferably, wherein the cancer, tumor, or cellular proliferation is selected from the group consisting of melanomas, lymphoma, T-cell leukemia, non-small cell lung carcinoma, hepatic carcinoma, gastric cancer, esophagus carcinomas, colorectal carcinomas, pancreatic endocrine neoplasms, ovarian neoplasms, cervical cancer, salivary glands carcinoma, head and neck squamous cell carcinomas, proligerating testes cells, spermatocytic seminoma, sporadic medullary thyroid carcinoma, osteosarcomas, childhood astrocytomas, bladder cancer, cells from inflamed joints in juvenile rheumatoid arthritis or other harmful inflammatory condition, glioma, neuroblastoma tumors, and cancers related to malignant mast cells.

The present invention further provides a method for treating a cancer, tumor, or cellular proliferation in a mammal, wherein the cancer or tumor comprises a cell that expresses a MAGE gene, the method comprising inhibiting the expression of the MAGE gene or inhibiting the function of a polypeptide encoded by the MAGE gene of the cell in the mammal.

The present invention also provides a pharmaceutical composition for treating tumor cell formation or tumor cell growth, comprising an antagonist to MAGE and a pharmaceutically acceptable excipient.

Also disclosed is a method for screening for a substance that inhibits MAGE gene expression or function, the method comprising: (1) providing a candidate substance to be tested; (2) applying said candidate substance to a cell expressing a MAGE gene or MAGE gene construct, (3) detecting the level of expression of the MAGE gene or MAGE gene construct in the presence of the candidate substance, and (4) determining a level of expression of the MAGE in the absence of the candidate substance, wherein a substance that decreases the level of expression of the MAGE gene is selected, or determining the ability of the MAGE gene product to bind to a target protein occurring naturally in the cell or expressed by recombinant technology

### DETAILED DESCRIPTION OF THE INVENTION

The present inventors discovered that MAGE gene expression promotes the proliferation and survival of malignant cells in which they are actively expressed. The present inventors observed that some MAGE family members are down regulated at both the mRNA and protein levels in malignant mast cells when apoptosis is induced in those malignant mast cells. Further investigation lead to the discovery that MAGE molecules have anti-apoptotic function and that interference with MAGE expression and/or function prevents proliferation and induces death in other tumor cells. Using MAGE-specific small interfering RNAs (siRNAs), the synthesis of MAGE proteins in tumor cells was prevented or decreased, and cultured malignant cells were killed and/or their growth suppressed.

Accordingly, the present invention in one embodiment provides a method for cancer treatment via inhibiting MAGE gene expression. In another embodiment, the present invention provides a method for cancer treatment via inhibiting MAGE protein function. The present invention in a further embodiment provides a method for screening for antagonists of MAGE gene products, which antagonists inhibit the expression of MAGE genes or the function of MAGE gene product.

The method of the present invention is applicable to any cancer or cellular proliferation known to be associated with expression of MAGE proteins. They include but are not limited to: melanomas, lymphoma, T-cell leukemia, non-small cell lung carcinoma, hepatic carcinoma, gastric cancer, esophagus carcinomas, colorectal carcinomas, pancreatic endocrine neoplasms, ovarian neoplasms, cervical cancer, salivary glands carcinoma, head and neck squamous cell carcinomas, spermatocytic seminoma, spermatogonia, testes cells, sporadic medullary thyroid carcinoma, osteosarcomas, childhood astrocytomas, bladder cancer, cells from inflamed joints in juvenile rheumatoid arthritis and other harmful inflammatory conditions, glioma, neuroblastoma tumors, and cancers related to malignant mast cells.

The method of the present invention is applicable to any member of any of numerous known MAGE families of genes. These genes include the Type I MAGE genes, including MAGE-A, MAGE-B, and MAGE-C. These genes are known to be expressed in a wide variety of histologically distinct tumors. MAGE-A further includes members known as MAGE-A1, A2, A3, A4, A5, A6, A8, A9, A10, A11 and A12, and MAGE-B further includes MAGE-B1, B2, B3 and B4. MAGE-C members include MAGE-C1, C2 and C3. Type II MAGE genes include the families of Necdin, MAGE-D (D1, also known as NRAGE) D2, D3 and D4, and MAGE-E (E1), MAGE-F, MAGE-G, MAGE-H and MAGE-L.

As discussed above, the functional role of most MAGE members remains uncharacterized. None of the MAGE genes is expressed in healthy adult tissues except male germ cells and placenta, so the expression patterns suggest that this gene family is involved in development and in the function of placenta and germ cells. However, many of the type I MAGE genes (MAGE-A, B, C) are expressed in a wide variety of histologically distinct tumors. The following paragraphs summarize what is known about MAGE gene function, based on in vitro experiments:

There are few reports in the literature that identify any role for MAGE genes in apoptosis and cell growth, and those few reports conflict regarding whether the MAGE genes stimulate or suppress growth and apoptosis in developing cells and in cancer cells. For instance, Necdin was reported to interact with cellular transcription factor E2F1, and act as a specific cell growth suppressor in early neurons. (Tanimuri et al., 1998, Necdin, a post mitotic neuron-specific growth suppressor, interacts with viral transforming proteins and cellular transcription factor E2F1, J. Biol. Chem. 273: 720-8.) However, another study by the same authors found that when both Necdin and the p53 protein were artificially over-expressed in osteosarcoma cells, Necdin could bind to p53. P53 is a nuclear transcription factor that affects expression of a number of proteins eventuating in regulation of cellular proliferation and apoptosis in some systems. In this system, over-expression p53 also induces cell cycle arrest and eventually apoptosis, and simultaneous over-expression of Necdin interferes with p53's ability to induce apoptosis. Thus Necdin appears to be one of a number proteins that can bind to p53 and block some or all of its functions, (Taninuri et al., 1999, Physical and functional interactions of neuronal growth suppressor necdin with p53. J. Biol. Chem. 274: 16242-8).

In addition, there are a number of studies that indicate that MAGE genes can induce apoptosis, or programmed cell death, teaching away from the present invention. For instance, NRAGE (MAGE-D1) has been implicated as pro-apoptotic, acting as a mediator of apoptosis (Barker et al., 1998, p75NTR: a study in contrasts. Cell Death Diff. 5:346-356. Barrett G L et al., 2000, The p75 neurotrophin receptor and neutonal apoptosis, Progr. Neurobiol. 61:205-229. Kapan, 2000, Neurotrophin signal transduction in the nervous system. Curr Opin Neurobiol 10:381-391. and Amir et al., 2000; Salehi et al., 2002, supra).

MAGE-A3 has been shown to regulate murine caspase-12, and when MAGE-A3 was transfected into muscle cells (RIKEN cell line C2C12) MAGE-A3 appeared to contribute to resistance of those cells to apoptosis induced by prolonged ER stress (Morishima et al., 2002, An endoplasmic reticulum stress-specific caspase cascade in apoptosis. Cytochrome c-independent activation of caspase-9 by caspase-12. J. Biol. Chem. 277:34287-94.). These studies, however, showed that inhibition of MAGE protein in these cells does not cause spontaneous apoptosis, because MAGE-A3 is not normally expressed in this murine muscle cell line. Furthermore, the anti-apoptosis effect of MAGE-A3 in these studies appear to be CASPASE-12 dependent, but the malignant cells that form the basis of our observations do not express caspase-12.

MAGE-A4 was shown to suppress the tumorogenic activity of the liver oncoprotein gankyrin ((Nagao et al, 2003, MAGE-A4 interacts with the liver oncoprotein gankyrin and suppresses its tumorigenic activity. J. Biol. Chem. 278:10668-74). In these studies, MAGE-A4 partially suppressed both anchorage-independent growth in vitro and tumor formation in athymic mice of gankyrin-overexpressing cells. MAGE-A4 did not affect the cell cycle progression, proliferation rate, nor apoptosis in these cells by itself. This observation is in contrast to the results obtained by the present inventors, which show that inhibition of MAGE A proteins induce tumor cell death.

Other studies have shown interactions of MAGE genes with specific proteins in vitro, but have not shown a specific relationship of the MAGE genes to apoptosis or cell proliferation. For instance, Ror2 (Ror family receptor tyrosine kinase) sequesters NRAGE in membranous compartments, thereby affecting the transcriptional function of Msx2 and possibly regulating limb morphogenesis (Matsuda et al, 2003, The receptor tyrosine kinase Ror2 associates with the melanoma-associated antigen (MAGE) family protein Dlxin-1 and regulates its intracellular distribution. J Biol. Chem. 278:29057-64.), and NRAGE (also known as Dlxin-1) interacts with Praja 1 thereby affecting the transcription function of the homeodomain protein Dlx5. (Sasaki et al, 2002, A RING finger protein Praja1 regulates Dlx5-dependent transcription through its ubiquitin ligase activity for the Dlx/Msx-interacting MAGE/Necdin family protein, Dlxin-1. J. Biol. Chem. 277:22541-6.)

The present invention provides antagonist compositions and methods that inhibit MAGE gene expression and function for the treatment or prevention of cancers or harmful cellular proliferations.

As the function or development of male germ cells also depends on the proper function of MAGE gene products, inhibition of MAGE genes in these cells will prevent them from developing or functioning normally. For example, Takahashi et al. (1995) identified MAGE-1 and MAGE-4 in the nucleus and cytoplasm of spermatogonia and in primary spermatocytes and concluded that MAGE proteins are normal tissue antigens compartmentalized in particular testicular cells playing an important role in the early phase of spermatogenesis (Cancer Res. 55:3478-3482). Chomez et al. (1995) showed that the sMAGE gene family is expressed in post-meiotic spermatids during mouse germ cell differentiation, suggesting that the SMAGE proteins play a role in the maturation of these spermatozoa (Immunogenetics 43:97-100). More recently, Gaskel et al. (2004) linked MAGE and KIT (c-KIT) in fetal testis, specifically that pre-spermatogonia are c-KIT negative and MAGE positive (Biology of reproduction 71:2012-2021). Inhibition of KIT is known to induce apoptosis of HMC-1 cells (Ma et al., 2002, The c-KIT mutation causing human mastocytosis is resistant to STI571 and other KIT kinase inhibitors; kinases with enzymatic site mutations show different inhibitor sensitivity profiles than wild-type kinases and those with regulatory-type mutations. Blood 99:1741-4.). Thus, a similar mechanism of action, i.e. KIT regulation of MAGE, may exist in developing sperms and our invention predicts that if MAGE expression is disrupted, apoptosis in sperm is triggered, leading to a male contraceptive.

Accordingly, an embodiment of the present invention provides contraceptive methods and compositions for male mammals, especially in man. Preferably, the contraceptive methods comprise administering to the mammal in need thereof a composition comprising one or more MAGE gene inhibitor, such as a small molecule antagonist, siRNA, an antibody against a MAGE gene product, or an antisense nucleic acid molecule, as will be discussed in more detail below.

In one embodiment, the composition may comprise reagents or factors that inhibit MAGE gene expression by regulating MAGE gene transcriptional activity. Such a composition may comprise reagents or factors that inhibit MAGE post-translational modification and its secretion if appropriate. Such a composition may comprise reagents that act as MAGE antagonists that block MAGE activity by competing with MAGE for binding to MAGE Partner Proteins, including but not limited to MAGE cell surface receptors. Alternatively, such a composition may comprise factors or reagents that inhibit the signaling pathway transduced by MAGE once bound to its receptors on or in cells.

In one embodiment, this invention provides neutralizing antibodies to inhibit MAGE biological action. In another embodiment of the invention, the MAGE antagonizing agents are antisense oligonucleotides to MAGE. The antisense oligonucleotides preferably inhibit MAGE expression by inhibiting translation of the MAGE protein. In a further embodiment, the antagonizing agent is small interfering RNAs (siRNA, also known as RNAi, RNA interference nucleic acids). siRNA are double-stranded RNA molecules, typically 21 n.t. in length, that are homologous to the target gene (e.g., MAGE) and interfere with the target gene's activity.

The antagonists of the invention (neutralizing and others) are preferably used as a treatment for cancer formation or growth. By the term “neutralizing” it shall be understood that the antagonists has the ability to inhibit or block the normal biological activity of MAGE or the binding of MAGE to Partner Proteins.

An anti-MAGE antibody suitable for the present invention may be a polyclonal antibody. Preferably, the antibody is a monoclonal antibody. The antibody may also be isoform-specific.

The monoclonal antibody or binding fragment thereof of the invention may be Fab fragments, F(ab)₂ fragments, Fab′ fragments, F(ab′)₂ fragments, Fd fragments, Fd′ fragments or Fv fragments. Domain antibodies (dAbs) (for review, see Holt et al., 2003, Trends in Biotechnology 21:484-490) are also suitable for the methods of the present invention.

Various methods of producing antibodies with a known antigen are well-known to those ordinarily skilled in the art (see for example, Harlow and Lane, 1988, Antibodies: A Laboratory Manual. Cold Spring Harbor Laboratory Press, Cold Spring Harbor, N.Y.; see also WO 01/25437). In particular, suitable antibodies may be produced by chemical synthesis, by intracellular immunization (i.e., intrabody technology), or preferably, by recombinant expression techniques. Methods of producing antibodies may further include the hybridoma technology well-known in the art.

In accordance with the present invention, the antibodies or binding fragments thereof may be characterized as those which are capable of specific binding to a MAGE protein or an antigenic fragment thereof, preferably an epitope that is recognized by an antibody when the antibody is administered in vivo. Antibodies can be elicited in an animal host by immunization with a MAGE-derived immunogenic component, or can be formed by in vitro immunization (sensitization) of immune cells. The antibodies can also be produced in recombinant systems in which the appropriate cell lines are transformed, transfected, infected or transduced with appropriate antibody-encoding DNA. Alternatively, the antibodies can be constructed by biochemical reconstitution of purified heavy and light chains.

The antibodies may be from humans, or from animals other than humans, preferably mammals, such as rat, mouse, guinea pig, rabbit, goat, sheep, and pig. Preferred are mouse monoclonal antibodies and antigen-binding fragments or portions thereof. In addition, chimeric antibodies and hybrid antibodies are embraced by the present invention. Techniques for the production of chimeric antibodies are described in e.g. Morrison et al., 1984, Proc. Natl. Acad. Sci. USA, 81:6851-6855; Neuberger et al., 1984, Nature, 312:604-608; and Takeda et al., 1985, Nature, 314:452-454.

Further, single chain antibodies are also suitable for the present invention (e.g., U.S. Pat. Nos. 5,476,786 and 5,132,405 to Huston; Huston et al., 1988, Proc. Natl. Acad. Sci. USA, 85:5879-5883; U.S. Pat. No. 4,946,778 to Ladner et al.; Bird, 1988, Science, 242:423-426 and Ward et al., 1989, Nature, 334:544-546). Single chain antibodies are formed by linking the heavy and light immunoglobulin chain fragments of the Fv region via an amino acid bridge, resulting in a single chain polypeptide. Univalent antibodies are also embraced by the present invention.

Many routes of delivery are known to the skilled artisan for delivery of anti-MAGE antibodies. For example, direct injection may be suitable for delivering the antibody to the site of interest. It is also possible to utilize liposomes with antibodies in their membranes to specifically deliver the liposome to the area of the tumor where MAGE expression or function is to be inhibited. These liposomes can be produced such that they contain, in addition to monoclonal antibody, other therapeutic agents, such as those described above, which would then be released at the tumor site (e.g., Wolff et al., 1984, Biochem. et Biophys. Acta, 802:259).

This invention also provides MAGE antisense nucleic acid molecules and compositions comprising such antisense molecules. The constitutive expression of antisense RNA in cells has been known to inhibit the gene expression, possibly via the blockage of translation or prevention of splicing. Interference with splicing allows the use of intron sequences which should be less conserved and therefore result in greater specificity, inhibiting expression of a gene product of one species but not its homologue in another species.

The term antisense component corresponds to an RNA sequence as well as a DNA sequence coding therefor, which is sufficiently complementary to a particular mRNA molecule, for which the antisense RNA is specific, to cause molecular hybridization between the antisense RNA and the mRNA such that translation of the mRNA is inhibited. Such hybridization can occur under in vivo conditions. This antisense molecule must have sufficient complementarity, about 18-30 nucleotides in length, to the MAGE gene so that the antisense RNA can hybridize to the MAGE gene (or mRNA) and inhibit MAGE gene expression regardless of whether the action is at the level of splicing, transcription, or translation. The antisense components of the present invention may be hybridizable to any of several portions of the target MAGE cDNA, including the coding sequence, 3′ or 5′ untranslated regions, or other intronic sequences, or to MAGE mRNA.

Antisense RNA is delivered to a cell by transformation or transfection via a vector, including retroviral vectors and plasmids, into which has been placed DNA encoding the antisense RNA with the appropriate regulatory sequences including a promoter to result in expression of the antisense RNA in a host cell. In one embodiment, stable transfection and constitutive expression of vectors containing MAGE cDNA fragments in the antisense orientation are achieved, or such expression may be under the control of tissue or development-specific promoters. Delivery can also be achieved by liposomes.

For in vivo therapy, the currently preferred method is direct delivery of antisense oligonucleotides, instead of stable transfection of an antisense cDNA fragment constructed into an expression vector. Antisense oligonucleotides having a size of 15-30 bases in length and with sequences hybridizable to any of several portions of the target MAGE cDNA, including the coding sequence, 3′ or 5′ untranslated regions, or other intronic sequences, or to MAGE mRNA, are preferred. Sequences for the antisense oligonucleotides to MAGE are preferably selected as being the ones that have the most potent antisense effects. Factors that govern a target site for the antisense oligonucleotide sequence include the length of the oligonucleotide, binding affinity, and accessibility of the target sequence. Sequences may be screened in vitro for potency of their antisense activity by measuring inhibition of MAGE protein translation and MAGE related phenotype, e.g., inhibition of cell proliferation in cells in culture. In general it is known that most regions of the RNA (5′ and 3′ untranslated regions, AUG initiation, coding, splice junctions and introns) can be targeted using antisense oligonucleotides.

The preferred MAGE antisense oligonucleotides are those oligonucleotides which are stable, have a high resistance to nucleases, possess suitable pharmacokinetics to allow them to traffic to target tissue site at non-toxic doses, and have the ability to cross through plasma membranes.

Phosphorothioate antisense oligonucleotides may be used. Modifications of the phosphodiester linkage as well as of the heterocycle or the sugar may provide an increase in efficiency. Phophorothioate is used to modify the phosphodiester linkage. An N3′-P5′ phosphoramidate linkage has been described as stabilizing oligonucleotides to nucleases and increasing the binding to RNA. Peptide nucleic acid (PNA) linkage is a complete replacement of the ribose and phosphodiester backbone and is stable to nucleases, increases the binding affinity to RNA, and does not allow cleavage by RNase H. Its basic structure is also amenable to modifications that may allow its optimization as an antisense component. With respect to modifications of the heterocycle, certain heterocycle modifications have proven to augment antisense effects without interfering with RNase H activity. An example of such modification is C-5 thiazole modification. Finally, modification of the sugar may also be considered. 2′-O-propyl and 2′-methoxyethoxy ribose modifications stabilize oligonucleotides to nucleases in cell culture and in vivo.

The delivery route will be the one that provides the best antisense effect as measured according to the criteria described above. In vitro cell culture assays and in vivo tumor growth assays using antisense oligonucleotides have shown that delivery mediated by cationic liposomes, by retroviral vectors and direct delivery are efficient. Another possible delivery mode is targeting using antibody to cell surface markers for the tumor cells. Antibody to MAGE or to its receptor may serve this purpose.

Alternatively, nucleic acid sequences which inhibit or interfere with gene expression (e.g., siRNA, ribozymes, aptamers) can be used to inhibit or interfere with the activity of RNA or DNA encoding MAGE.

siRNA technology relates to a process of sequence-specific post-transcriptional gene repression which can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Accordingly, siRNA may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the siRNA (sequence-specific) pathway, the initiating dsRNA is first broken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length.

The nonspecific effects occur because dsRNA activates two enzymes: PKR, which in its active form phosphorylates the translation initiation factor elF2 to shut down all protein synthesis, and 2′, 5′ oligoadenylate synthetase (2′,5′-AS), which synthesizes a molecule that activates RNase L, a nonspecific enzyme that targets all mRNAs. The nonspecific pathway may represent a host response to stress or viral infection, and, in general, the effects of the nonspecific pathway are preferably minimized. Significantly, longer dsRNAs appear to be required to induce the nonspecific pathway and, accordingly, dsRNAs shorter than about 30 bases pairs are preferred to effect gene repression by RNAi (see Hunter et al., 1975, J. Biol. Chem. 250:409-17; Manche et al., 1992, Mol. Cell. Biol. 12:5239-48; Minks et al., 1979, J. Biol. Chem. 254:10180-3; and Elbashir et al., 2001, Nature 411:494-8). siRNA has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see Bass, 2001, Nature 411:428-9). In mammalian cells, siRNAs are effective at concentrations that are several orders of magnitude below the concentrations typically used in antisense experiments (Elbashir et al., 2001, Nature 411:494-8).

The double stranded oligonucleotides used to effect RNAi are preferably less than 30 base pairs in length and, more preferably, comprise about 25, 24, 23, 22, 21, 20, 19, 18 or 17 base pairs of ribonucleic acid. Optionally the dsRNA oligonucleotides may include 3′ overhang ends. Exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al., 2001, Nature 411:494-8).

Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable to the skilled artisan.

Exemplary dsRNAs may be synthesized chemically or produced in vitro or in vivo using appropriate expression vectors. Exemplary synthetic RNAs include 21 nucleotide RNAs chemically synthesized using methods known in the art. Synthetic oligonucleotides are preferably deprotected and gel-purified using methods known in the art (see e.g. Elbashir et al., 2001, Genes Dev. 15:188-200). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species will be designed to include a portion of nucleic acid sequence represented in a MAGE nucleic acid.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, the contents of which are incorporated herein by reference.

Although mRNAs are generally thought of as linear molecules containing the information for directing protein synthesis within the sequence of ribonucleotides, most mRNAs have been shown to contain a number of secondary and tertiary structures. Secondary structural elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see e.g. Jaeger et al., 1989, Proc. Natl. Acad. Sci. USA 86:7706; and Turner et al., 1988, Annu. Rev. Biophys. Biophys. Chem. 17:167). The rules are useful in identification of RNA structural elements and, in particular, for identifying single stranded RNA regions which may represent preferred segments of the mRNA to target for siRNA, ribozyme or antisense technologies. Accordingly, preferred segments of the mRNA target can be identified for design of the siRNA mediating dsRNA oligonucleotides as well as for design of appropriate ribozyme and hammerhead ribozyme compositions of the invention (see below).

The dsRNA oligonucleotides may be introduced into the cell by transfection with a heterologous target gene using carrier compositions such as liposomes, which are known in the art—e.g. Lipofectamine 2000 (Life Technologies) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP-encoding pAD3 (Kehlenback et al., 1998, J. Cell Biol. 141:863-74). The effectiveness of the siRNA may be assessed by any of a number of assays following introduction of the dsRNAs. These include Western blot analysis using antibodies which recognize the MAGE gene product following sufficient time for turnover of the endogenous pool after new protein synthesis is repressed, reverse transcriptase polymerase chain reaction and Northern blot analysis to determine the level of existing MAGE target mRNA.

As early as in 2003, lipid-mediated systemic delivery of siRNAs was known to be effective and beneficial and for the treatment of cancer. For example, Sioud and Sorensen (2003, Biochemical and Biophysical Research Communications 312:1220-1225), recognized that a major obstacle to the use of siRNAs as therapeutics is the difficulty involved in effective in vivo delivery. Using a fluorescein-labeled siRNA, they investigated cationic liposome-mediated intravenous and intraperitoneal delivery in adult mice, and showed that this simple approach was able to deliver siRNAs into various cell types. In addition, they showed that in contrast to mouse cells, siRNAs can activate the non-specific pathway in human freshly isolated monocytes, resulting in TNF-α and IL-6 production. Their data also indicate that certain siRNA sequences can activate the innate immunity response genes.

Urban-Klein et al. (2005, Gene Therapy 12:461-466. (Published online 23 Dec. 2004)) established an effective and simple system for the systemic in vivo application of siRNAs as a powerful tool for therapeutic use through low molecular weight polyethylenimine (PEI) complexation. They showed that non-covalent complexation of synthetic siRNAs with PEI efficiently stabilizes siRNAs and delivers siRNAs into cells where they display full bioactivity at completely nontoxic concentrations. More importantly, in a subcutaneous mouse tumor model, the systemic (intraperitoneal, i.p.) administration of complexed, but not of naked siRNAs, leads to the delivery of the intact siRNAs into the tumors. The i.p. injection of PEI-complexed, but not of naked siRNAs targeting the c-erbB2/neu (HER-2) receptor results in a marked reduction of tumor growth through siRNA-mediated HER-2 down-regulation.

Yano et al. (2004, Clinical Cancer Research 10: 7721-7726) devised a method that complexed a synthetic siRNA, B717, which is sequence specific for the human bcl-2 oncogene, with a novel cationic liposome, LIC-101. The method overcomes the difficulties of the hydrodynamic method usually used for rapid administration of oligonucleotides which is generally unsuitable for use in humans. They investigated the antitumor activity of the complexed siRNA using a mouse model of liver metastasis. B717/LIC-101 was administered by bolus intravenous injection, where the rate and volume of administration were adjusted to what is feasible in human therapy. They also used a mouse model bearing prostate cancer in which the cells were inoculated under the skin, and administered B717/LIC-101 subcutaneously around the tumor. They discovered that the B717/LIC-101 complex inhibited the expression of bcl-2 protein and the growth of tumor cell lines in vitro in a sequence-specific manner in the concentration range of 3 to 100 nmol/L. Furthermore, the complex had a strong antitumor activity when administered intravenously in the mouse model of liver metastasis. B717 (siRNA) was shown to be delivered to tumor cells in the mouse liver, but only when complexed with LIC-101. The complex also inhibited tumor cell growth in the mouse model bearing prostate cancer.

Duxbury et al. (2004, Ann Surg. 240:667-676, Systemic siRNA-mediated gene silencing: a new approach to targeted therapy of cancer) evaluated the ability of systemically administered siRNA to silence gene expression in vivo and assessed the effect of the approach on tumor growth using a murine pancreatic adenocarcinoma xenograft model. The data in the publication demonstrate the efficacy of systemically administered siRNA as a therapeutic modality in experimental pancreatic cancer, and suggest that systemically administered siRNA is applicable to a broad range of cancers including patients with refractory disease. They used the carcinoembryonic antigen-related cell adhesion molecule 6 (CEACAM6), which is widely overexpressed in human gastrointestinal cancer, and its overexpression is known to promote cell survival under anchorage independent conditions, a characteristic associated with tumorigenesis and metastasis. Using real-time polymerase chain reaction (PCR) and Western blot, they quantified CEACAM6 expression. Mice (n=10/group) were subcutaneously xenografted with 2×10⁶ BxPC3 cells (which inherently overexpress CEACAM6). Tumor growth, CEACAM6 expression, cellular proliferation (Ki-67 immunohistochemistry), apoptosis, angiogenesis (CD34 immunohistochemistry), and survival were compared for mice administered either systemic CEACAM6-specific or control single-base mismatch siRNA over 6 weeks, following orthotropic tumor implantation. The results showed that treatment with CEACAM6-specific siRNA suppressed primary tumor growth by 68% versus control siRNA (P<0.05) and was associated with a decreased proliferating cell index, impaired angiogenesis and increased apoptosis in the xenografted tumors. CEACAM6-specific siRNA completely inhibited metastasis (0% of mice versus 60%, P<0.05) and significantly improved survival, without apparent toxicity.

Further compositions, methods and applications of siRNA technology are provided in U.S. Pat. Nos. 6,278,039, 5,723,750 and 5,244,805, which are incorporated herein by reference.

MAGE gene expression can also be inhibited or disrupted by genetically altering the coding regions of the gene or regulatory sequences that control its expression. For example, the promoter of a MAGE gene can be disrupted using homologous recombination to insert a disrupting nucleotide sequence into the genome of a target mammalian cell. Using one or two regions homologous to the promoter of a target gene sequence, a nucleotide can be incorporated via homologous recombination into the promoter region, thereby disrupting the promoter. Similarly, the coding regions, including both the introns and exons, can be targeted for disruption via homologous recombination. The DNA is stably incorporated into the genome of a cell. For more details, please U.S. Pat. No. 5,272,071 and U.S. Pat. No. 5,641,670, which are incorporated herein by reference in their entirety.

Ribozymes are enzymatic RNA molecules capable of catalyzing specific cleavage of RNA. (For a review, see Rossi, 1994, Current Biology 4:469-471). The mechanism of ribozyme action involves sequence specific hybridization of the ribozyme molecule to complementary target RNA, followed by an endonucleolytic cleavage event. The composition of ribozyme molecules preferably includes one or more sequences complementary to a MAGE mRNA, and the well known catalytic sequence responsible for mRNA cleavage or a functionally equivalent sequence (see, e.g., U.S. Pat. No. 5,093,246, which is incorporated herein by reference in its entirety). Ribozyme molecules designed to catalytically cleave MAGE mRNA transcripts can also be used to prevent translation of subject MAGE mRNAs.

While ribozymes that cleave mRNA at site-specific recognition sequences can be used to destroy target mRNAs, the use of hammerhead ribozymes is preferred. Hammerhead ribozymes cleave mRNAs at locations dictated by flanking regions that form complementary base pairs with the target mRNA. Preferably, the target mRNA has the following sequence of two bases: 5′-UG-3′. The construction and production of hammerhead ribozymes is well known in the art and is described more fully in Haseloff and Gerlach, 1988, Nature 334:585-591; and PCT Application. No. WO89/05852, the contents of which are incorporated herein by reference. Hammerhead ribozyme sequences can be embedded in a stable RNA such as a transfer RNA (tRNA) to increase cleavage efficiency in vivo (Perriman et al., 1995, Proc. Natl. Acad. Sci. USA, 92:6175-79; de Feyter, and Gaudron, Methods in Molecular Biology, Vol. 74, Chapter 43, “Expressing Ribozymes in Plants”, Edited by Turner, P. C, Humana Press Inc., Totowa, N.J.). In particular, RNA polymerase III-mediated expression of tRNA fusion ribozymes are well known in the art (see Kawasaki et al., 1998, Nature 393:284-9; Kuwabara et al., 1998, Nature Biotechnol. 16:961-5; and Kuwabara et al., 1998, Mol. Cell 2:617-27; Koseki et al., 1999, J. Virol. 73:1868-77; Kuwabara et al., 1999, Proc. Natl. Acad. Sci. USA, 96:1886-91; Tanabe et al., 2000, Nature 406:473-4). There are typically a number of potential hammerhead ribozyme cleavage sites within a given target cDNA sequence. Preferably the ribozyme is engineered so that the cleavage recognition site is located near the 5′ end of the target mRNA- to increase efficiency and minimize the intracellular accumulation of non-functional mRNA transcripts. Furthermore, the use of any cleavage recognition site located in the target sequence encoding different portions of the MAGE mRNA would allow the selective targeting of one or the other MAGE genes.

Gene targeting ribozymes necessarily contain a hybridizing region complementary to two regions, each of at least 5 and preferably each 6, 7, 8, 9, 10, 11, 12, 13, 14, 15, 16, 17, 18, 19 or 20 contiguous nucleotides in length of a MAGE mRNA. In addition, ribozymes possess highly specific endoribonuclease activity, which autocatalytically cleaves the target sense mRNA.

The ribozymes of the present invention also include RNA endoribonucleases (“Cech-type ribozymes”) such as the one which occurs naturally in Tetrahymena thermophila (known as the IVS, or L-19 IVS RNA) and which has been extensively described in Zaug et al., 1984, Science, 224:574-578; Zaug et al., 1986, Science 231:470-475; Zaug et al., 1986, Nature 324:429-433; published International patent application No. WO88/04300; and Been et al., 1986, Cell 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in a target gene or nucleic acid sequence.

Ribozymes can be composed of modified oligonucleotides (e.g., for improved stability, targeting, etc.) and should be delivered to cells which express the target gene in vivo. A preferred method of delivery involves using a DNA construct “encoding” the ribozyme under the control of a strong constitutive promoter, so that transfected cells will produce sufficient quantities of the ribozyme to destroy endogenous target messages and inhibit translation. Because ribozymes, unlike antisense molecules, are catalytic, a lower intracellular concentration is required for efficiency.

In certain embodiments, a ribozyme may be designed by first identifying a sequence portion sufficient to cause effective knockdown by RNAi. The same sequence portion may then be incorporated into a ribozyme. In this aspect of the invention, the gene-targeting portions of the ribozyme or siRNA are substantially the same sequence of at least 5 and preferably 6, 7, 8, 9, 10, 11, 12, 13, 14, 15, 16, 17, 18, 19 or 20 or more contiguous nucleotides of a MAGE nucleic acid.

In a long target RNA chain, significant numbers of target sites are not accessible to the ribozyme because they are hidden within secondary or tertiary structures (Birikh et al., 1997, Eur. J. Biochem. 245:1-16). To overcome the problem of target RNA accessibility, computer generated predictions of secondary structure are typically used to identify targets that are most likely to be single-stranded or have an “open” configuration (see Jaeger et al., 1989, Methods Enzymol. 183:281-306). Other approaches utilize a systematic approach to predicting secondary structure which involves assessing a huge number of candidate hybridizing oligonucleotides molecules (see Milner et al., 1997, Nat. Biotechnol. 15: 537-41; and Patzel and Sczakiel, 1998, Nat. Biotechnol. 16:64-8). Additionally, U.S. Pat. No. 6,251,588, the contents of which are herein incorporated by reference, describes methods for evaluating oligonucleotide probe sequences so as to predict the potential for hybridization to a target nucleic acid sequence. The method of the invention provides for the use of such methods to select preferred segments of a target mRNA sequence that are predicted to be single-stranded and, further, for the opportunistic utilization of the same or substantially identical target mRNA sequence, preferably comprising about 10-20 consecutive nucleotides of the target mRNA, in the design of both the siRNA oligonucleotides and ribozymes of the invention.

Alternatively, MAGE gene expression can be reduced by targeting deoxyribonucleotide sequences complementary to the regulatory region of the gene (i.e., the promoter and/or enhancers) to form triple helical structures that prevent transcription of the gene in target cells in the body. (See generally, Helene, C., 1991, Anticancer Drug Des., 6:569-84; Helene, C. et al., 1992, Ann. N.Y. Acad. Sci., 660:27-36; and Maher, L. J., 1992, Bioassays 14:807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the MAGE sequences that can be targeted for triple helix formation may be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

A further aspect of the invention relates to the use of DNA enzymes to inhibit expression of MAGE gene. DNA enzymes incorporate some of the mechanistic features of both antisense and ribozyme technologies. DNA enzymes are designed so that they recognize a particular target nucleic acid sequence, much like an antisense oligonucleotide. They are, however, catalytic and specifically cleave the target nucleic acid.

There are currently two basic types of DNA enzymes, both of which were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that will target the enzyme to the message is divided so that it comprises the two arms of the DNA enzyme, and the DNA enzyme loop is placed between the two specific arms.

Methods of making and administering DNA enzymes can be found, for example, in U.S. Pat. No. 6,110,462. Similarly, methods of delivery DNA ribozymes in vitro or in vivo are similar methods of delivery RNA ribozyme, as outlined in detail above. Additionally, one of skill in the art will recognize that, like antisense oligonucleotide, DNA enzymes can be optionally modified to improve stability and improve resistance to degradation.

The dosage ranges for the administration of the antagonists of the invention are those large enough to produce the desired effect. The dosage should not be so large as to cause adverse side effects, such as unwanted cross-reactions, anaphylactic reactions, and the like. Generally, the dosage will vary with the age, condition, sex and extent of disease of the patient and can be determined by one of skill in the art. The dosage can be adjusted by the individual physician in the event of any complication.

The antagonists of the invention can be administered parenterally by injection or by gradual perfusion over time. The antagonists can be administered intravenously, intraperitoneally, intramuscularly, subcutaneously, intracavity, or transdermally.

Another embodiment of the present invention relates to pharmaceutical compositions comprising one or more antagonists, according to the invention, together with a physiologically- and/or pharmaceutically-acceptable carrier, excipient, or diluent. Physiologically acceptable carriers, excipients, or stabilizers are known to those skilled in the art (see Remington's Pharmaceutical Sciences, 17th edition, (Ed.) A. Osol, Mack Publishing Company, Easton, Pa., 1985). Acceptable carriers, excipients or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

A further embodiment of the present invention relates to a method for screening for a substance that inhibits MAGE gene expression, the method comprising: (1) providing a candidate substance to be tested; (2) applying said candidate substance to a cell expressing a MAGE gene or MAGE gene construct, (3) detecting the level of expression of the MAGE gene or MAGE gene construct in the presence of the candidate substance, and (4) determining a level of expression of the MAGE in the absence of the candidate substance, wherein a substance that decreases the level of expression of the MAGE gene is selected.

The specific mechanism by which MAGE family proteins promote cancer cell survival is likely mediated through direct physical contact between the MAGE protein and a partner protein or protein. Such partner protein(s) may be identified using a yeast or mamalian two-hybrid assay with a MAGE protein as the bait. Therefore, the basis for one type of assay of MAGE function is to test for the ability of a substance to interfere with the ability of the MAGE protein to bind to a specific partner protein. In one example, we will use a yeast or mammalian cell two hybrid assay with MAGE protein and the partner(s) identified in our original yeast two hybrid screens. These two hybrid assays are based on the fact that transcriptional activators may consist of two functional domains, and that these two domains may be physically separate on two different proteins, provided that the proteins can bind to each other. One of the two domains is a DNA binding domain (BD) that specifically binds to a gene promoter or other cis regulatory element. The other domain is an activating domain (AD) that directs an RNA polymerase to transcribe a reporter gene that lies immediately downstream of the promoter region. An example of such a system is the Mammalian Mathmaker Two-Hybrid assay kit available commercially from Clontech Laboratories, Inc. For this assay we will subclone MAGE and partner protein cDNAs into expression plasmids that produce proteins that are a fusion of the MAGE gene and the GAL-4 DNA binding domain or are a fusion of the partner protein and the VP-16 activating domain. Normally, when these two plasmids are co-transfected with a reporter plasmid containing a CAT reporter gene construct downstream of the GAL4-BD binding site and the VP16-AD activation site, the binding of the MAGE protein to the Partner Protein will bring the BD and AD into proximity, thereby allowing activation of the CAT reporter gene, which is then detected by a CAT assay. Any substance that inhibits the binding of the MAGE protein to the Partner Protein would be detectable because it would turn the CAT reporter gene off. Other two-hybrid systems and other reporter genes could be used.

In other examples, in vitro assays detecting binding of MAGE to a Partner Protein could use conjugation of MAGE and the Partner Protein to fluorochromes and fluorochrome enhancers or quenchers, the so called “molecular beacon” technique. In these techniques, the amount of fluorescent signal from the labeled MAGE or Partner Protein is either enhance or quenched by binding of the MAGE and Partner proteins which brings the flurochrome and the enhancer or quencher into close proximity. Interference with MAGE-Partner protein binding separates the flurochrome and the quencher or enhancer, resulting in a change in the signal intensity of the flurochrome and identification of a candidate substance.

A further assay is based on the ability of a substance to inhibit activation of a MAGE gene or the function of a MAGE gene on the level of transcription or translation. This assay would use one of the many common techniques for detection of specific functional MAGE gene mRNA. In one example, the promoter region sequences, immediate downstream sequences, and sequences encoding transcription initiation and translation initiation regions of the MAGE gene are fused with a reporter gene cDNA such as the CAT reporter gene coding region. This construct is stably transfected into a MAGE expressing cell line and the resulting cell line are used to screen for molecules that interfere with expression of the reporter protein. A cell-free system could also be used, once the appropriate transcription factors are known.

Still another type of assay would detect the ability of a substance to inhibit MAGE protein enzymatic activity, or enzymatic activity of a Partner Protein, should such enzymatic activity be detected. This type of assay would use classic or new enzyme-substrate assay techniques with naturally occurring or synthetic substrates.

### EXAMPLES

### Example 1

### Array Analysis

The human mast cell line HMC-1 is dependent on the expression of mutant KIT protein which is constitutively activated, and inhibition of activated KIT with a drug induces apoptosis of HMC-1 cells (Ma et al., 2002, The c-KIT mutation causing human mastocytosis is resistant to STI571 and other KIT kinase inhibitors; kinases with enzymatic site mutations show different inhibitor sensitivity profiles than wild-type kinases and those with regulatory-type mutations. Blood 99:1741-4.). However, the mechanism by which KIT inhibitors cause apoptosis in these cells is unknown. To look for genes whose products could regulate and transduce KIT related signals controlling apoptosis, we used gene chip array analysis to study changes in the mRNA levels of specific genes following induction of apoptosis. These studies showed for the first time that HMC-1 cells express MAGE family proteins, and that the genes encoding these proteins were down regulated when apoptosis was induced, suggesting that decreasing the levels of expression of MAGE genes can induce apoptosis.

HMC-1.1 cells were divided into triplicate cultures of two groups: a control group treated with PBS, and an experimental group treated with 1 μM STI571, a KIT inhibitor for 4 hours. A quarter of the cells were used for Western blots to confirm the KIT inhibition, and the rest were used for total RNA isolation for genechip analysis. We used Affymetrix Genechip human Genome U133A chips. The arrays were hybridized and scanned and the data were analyzed using dChip analysis software. The statistical significance of differences in mRNA expression levels between STI571 treated and control cells were assessed by t-test based on data from three independent replicates. Values are expressed as Means +/−SEM, and p<0.05 was considered statistically significant.

TABLE 1
Down-regulation MAGE mRNA by STI 571 in HMC-1.1 cell

| Gene Description | GenBank Accession No. | Control | STI 571 |
|---|---|---|---|
| melanoma antigen, family A, 1 | NM_004988 | 757 ± 29 | 562 ± 33 |
| (directs expression of antigen MZ2-E) | | | |
| melanoma antigen, family A, 3 | BC000340 | 977 ± 67 | 779 ± 32 |
| melanoma antigen, family A, 5 | AI200443 | 520 ± 28 | 414 ± 28 |
| melanoma antigen, family A, 6 | U10691 | 896 ± 54 | 712 ± 42 |
| melanoma antigen, family A, 12 | BC003408 | 513 ± 29 | 410 ± 26 |
| melanoma antigen, family E, 1 | NM_016249 | 514 ± 13 | 405 ± 21 |

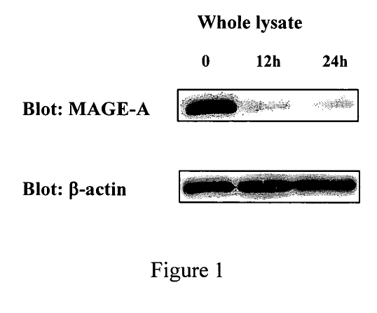

FIG. 1 shows a Western blot result of MAGE-A protein level in HMC-1.1 cells at 12 and 24 hours after the administration of STI 571.

The above results indicate that STI 571 causes a decrease of MAGE mRNA and protein in the cells, which is followed by apoptosis of the cells.

### Example 2

### MAGE siRNA Significantly Inhibited Growth of Various Cancer Cells

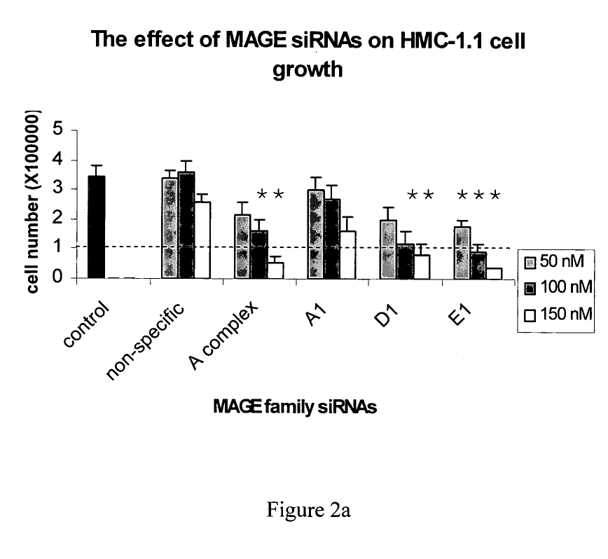

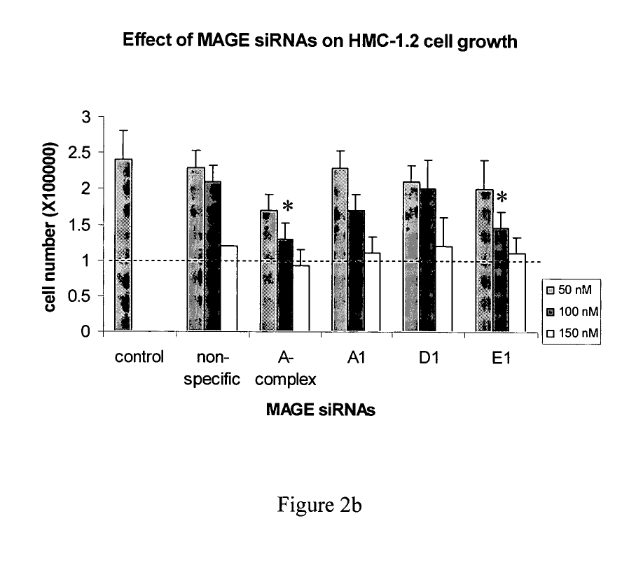

As shown in FIGS. 2a and 2b, both MAGE-D1 and MAGE-A siRNAs significantly inhibited growth of HMC-1.1 malignant mast cell subclone at 50 nM and 100 nM. MAGE-E1 siRNA showed significant inhibition of HMC-1.1 cell growth at 50 nM, and MAGE-A and MAGE-E1 significantly inhibited growth of the HMC-1.2 malignant mast cell subclone growth at 100 nM.

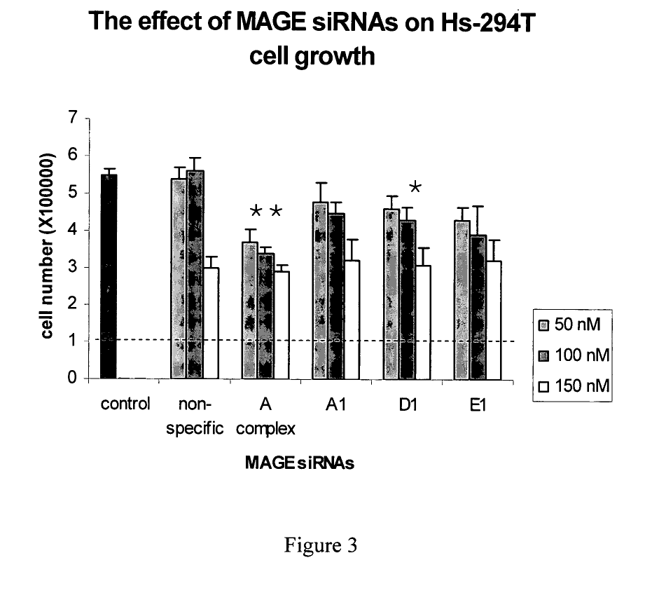

To show that this is a general effect, and to increase the potential types of tumors that could be treated, we looked at the effect of MAGE specific siRNAs on the growth of cultured human melanoma cells. The human melanoma line Hs-294T was inhibited by MAGE-A specific siRNA at 50 nM and by MAGE-D1 specific siRNA at 100 nM (FIG. 3).

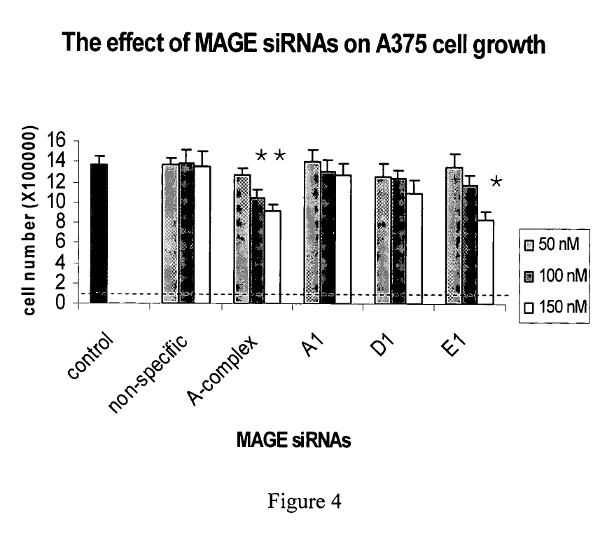

Another human melanoma line, A-375, was inhibited by MAGE-A specific siRNA 100 nM and MAGE-E1 specific siRNA at 150 nM (FIG. 4).

Non-specific (randomly synthesized) siRNA used as a control did not inhibit the growth of the HMC-1 cells or the two human melanoma lines (FIGS. 2-4).

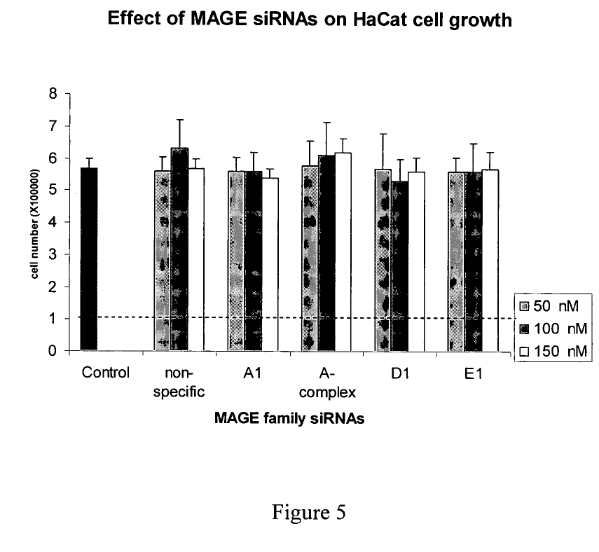

Furthermore, none of the siRNAs (both nonspecific and specific siRNAs), showed adverse affects on a human epithelial cancer cell line, HaCaT, which does not express these MAGE proteins (FIG. 5). This observation confirms the specificity of the effect by showing that neither the transfection process nor the non-specific siRNAs kill the cells at the siRNA concentrations used.

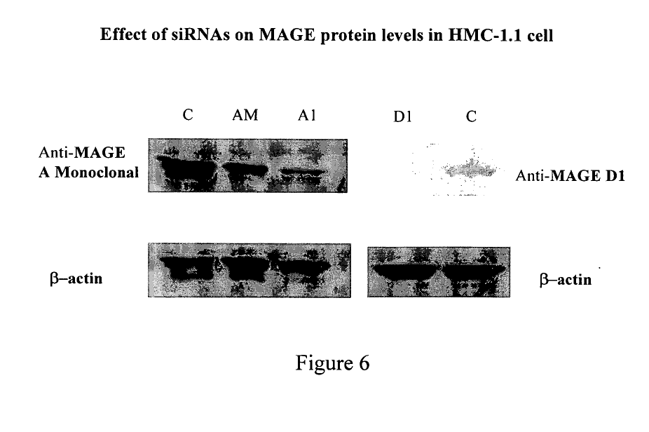

To further confirm that the effective of the siRNA was mediated through inhibition of MAGE proteins, we performed western blots on HMC-1.1 cells transfected with siRNA and found that the specific siRNA reagents effectively prevented the synthesis of the corresponding MAGE proteins but that non-specific siRNA reagents did not effect MAGE protein synthesis (FIG. 6).

### Example 3

### In Vivo Studies to Demonstrate that the Effectiveness of the Method of the Present Invention

The usefulness of synthetic siRNAs in inhibiting the growth of mast cells and melanoma cells is tested in in vivo mouse models of aggressive mastocytosis using the P815 murine malignant mast cell line and melanoma using the S91 Cloudman murine melanoma cell line. Both cell lines were originally derived from DBA/2 mice, and are therefore congenic with DBA/2 mice. 1.5×10⁶ S91 melanoma cells or 1.5×10⁵ P815 mast cells are injected into the peritoneal cavity or subcutaneously into DBA/2 mice in a volume of 0.2 ml. In some experiments the cells are treated with siRNA or other inhibitor, or vehicle, before injection. The mice are then randomly allocated to treated and untreated (control) groups.

Starting on the day of induction of the tumor appearance or day of subcutaneous tumor cell injection the mice are given daily intraperitoneal (IP.) injections of up to 10 mg/kg body weight of siRNA diluted in 100 μl of sterile PBS or cationinc lipid.

The survival rate for untreated animals ranges from 20-25 days and the effectiveness of the method of the present invention is further demonstrated as the treated animals live longer, on average, than the untreated (control) group of animals.

### Example 4

### Mice Inoculated with MAGE siRNA-Transfected Tumor Cells Showed Slower Tumor Growth Compared to Mice Inoculated with Non-Transfected Tumor Cells or Cells Transfected Non-Specific siRNA

S91 cloudman melanoma cells (1×10⁵) were seeded in a 6 well plate with 2 ml of F-12K (ATCC) medium containing 15% horse serum and 2.5% fetal bovine serum 24 hours pre-transfection. The medium was replaced with fresh medium without serum or antibiotics, and cells were transfected with 0.1 nmol of control siRNA or mMAGE-b siRNA in lipofectamine 2000™ (Invitrogen) according to the manufacture's instructions, to a final concentration of 100 nM siRNA 1 ml final volume. After 4 hours, 2× horse and fetal bovine serum were added to reach a normal culture condition. Eight hours after transfection, the cells were harvested, washed and re-suspended in Hanks' balanced salt solution to achieve a concentration of 2.5×10⁶ cells/ml. One hundred microliters of cell solution (2.5×10⁵ cells) were inoculated subcutaneously in the flank of mice, and tumors were measured every other day. Tumors were measured every other day in at least two dimensions (x and y), and mice were sacrificed when any dimension of the tumor reached 15 mm. The mean diameter of the tumor is calculated as √(x*y), which was used to evaluate the tumor growth. All of data are presented as √(x*y).

The time for a tumor to reach the target mean tumor diameter of 13 mm is defined as the elapsed time from the date of cell implantation to the date when a 13 mm target is reached, or when the mouse was sacrificed which is considered censored. Kaplan-Meier survival analysis with the corresponding Log-Rank analysis was performed using S-plus Software (Insightful; Seattle, Wash.). Linear Regression analysis was used to measure the rate of mean tumor diameter growth as a function of time using S-plus Software (Insightful; Seattle, Wash.). A p-value of >0.01 was considered to be statistically significant.

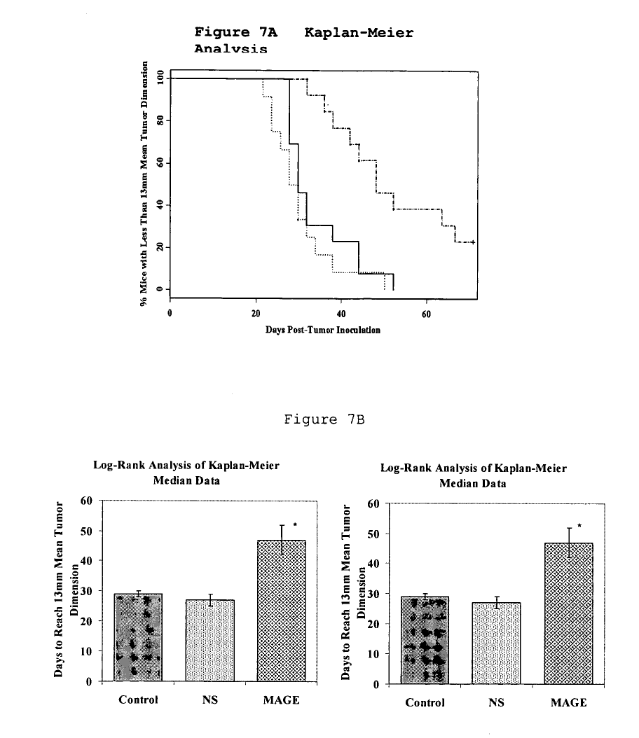

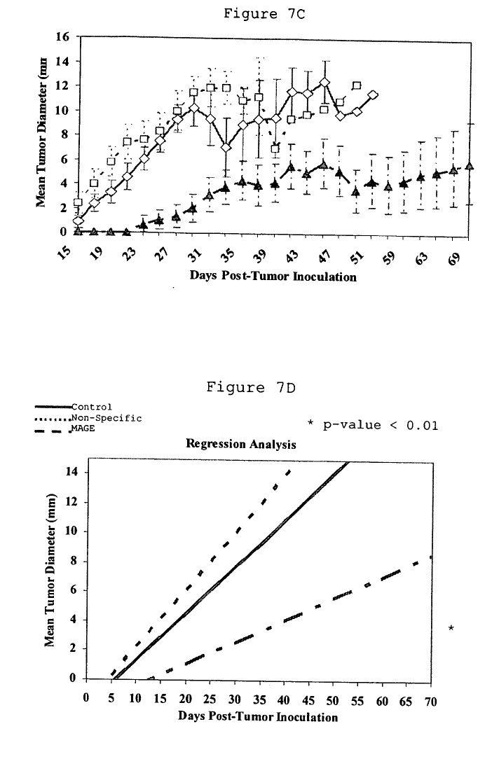

Thirty-six (36) mice were used in the study, of which 10 were inoculated with untreated cells (control), and 13 each were inoculated with cells treated with nonspecific siRNA and 13 with cells treated with MAGE siRNA. By day 51, all of the control mice inoculated with untreated cells, 12 of the 13 mice inoculated with cells treated with nonspecific siRNA (one died of unknown causes, probably trauma), and 8 of the 13 mice inoculated with cells treated with MAGE siRNA, (30 in total) reached the tumor target mean diameter of 13 mm. The average time to a mean tumor diameter of 13 mm was 33 days post-inoculation among the control mice, 30 days for mice treated with nonspecific siRNA, and 45 days for mice treated with specific MAGE siRNA. FIG. 7a shows a Kaplan-Meier plot comparing MAGE siRNA treated tumors to tumors treated with non-specific siRNA and untreated (control) tumors. FIG. 7b compares MAGE siRNA treated tumors to control and nonspecific siRNA treated tumors by Log-Rank analysis. As shown in FIG. 7b, tumor growth was significantly (p<0.01) slower in MAGE siRNA treated tumors when compared to both control mice and mice treated with non-specific siRNA.

FIG. 7c plots the daily tumor size data, and FIG. 7d is the Linear Regression analysis of the same data. FIG. 7d shows that tumors grew an average of 0.32 mm per day post-inoculation in the control group, 0.38 mm per day in the nonspecific group, and only 0.15 mm per day for the MAGE group. The observed differences for MAGE siRNA treatment as compared with either control or nonspecific siRNA treatments were statistically significant with p<0.01.

### Example 5

### Inhibiting Mage Expression Via Mage siRNa in Mice Inoculated with Tumor Cells Slows Tumor Growth

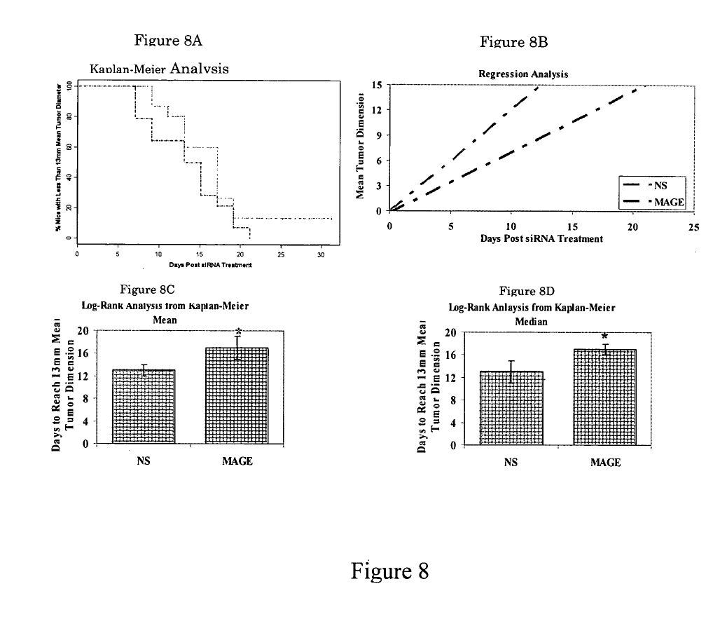

Male DBA/2 mice (8-week old) were obtained from Jackson Labs (Bar Harbor Me.), kept in filter topped cages with standard rodent chow and water available ad libitum, and a 12 h light/dark cycle. The experiments were performed according to national regulations and approved by the local animal care and use committee. Subcutaneous melanoma tumors were induced by inoculation of 2.5×10⁵ S91 cells in the left flank of mice. Mice were checked every other day by palpation, and when a tumor volume of 0.5 cm³ was reached, mice were randomized to receive either 0.6 nmol of control or MAGE-b siRNA by intra-tumor (IT) or intra-peritoneal (IP) injection of 0.1 ml of a solution containing in vivo Jet-PEI (Avanti Polar Lipids, Inc., Alabaster, Ala.), at an N/P (which is defined as the number of nitrogen residues of jetPEI™ per DNA phosphate. Zanta et al., 1997, In-Vitro Gene Delivery to Hepatocytes with Galactosylated Polyethylenimine. Bioconj. Chem. 8, 839-844) of 6. The mice were injected every other day for total of 10 injections. Tumors were measured every other day, and mice were sacrificed when any dimension of the tumor reached 15 mm (or an average diameter of 13 mm). Results are shown in FIG. 8 and demonstrate that parenteral MAGE siRNA administration significantly slowed down tumor growth compared with control or non-specific siRNA.

### Example 5

### Method for Screening for Small Molecule Mage Inhibitors

In a mammalian two-hybrid assay, cDNAs encoding proteins of interest are cloned into complimentary expression plasmids to create two complimentary fusion proteins (hybrids). If these two fusion proteins bind to each other, they will produce a detectable signal when the plasmids are co-transfected into cell lines with a third plasmid that encode an appropriate reporter gene. Transfected cells are grown in the presence of compounds from a compound library and when one of the compounds interferes with MAGE binding the reporter gene signal is lost. Compounds are then tested for their ability to induce apoptosis in melanoma cells or other cells.

Libraries of small molecules are screened for compounds with the ability to block MAGE binding, using a variant of an established mammalian two hybrid binding assay in the Small Molecule Screening Facility (SMSF), a joint venture of the UW Madison Comprehensive Cancer Center and the UW Keck Center for Chemical Genomics.

The SMSF provides a complete service including: screening and liquid handling equipment, informatics for data handling and analysis, assay design expertise, and centralized storage of compound libraries. The SMSF has robotic equipment and chemical libraries for automated screening using cell-based and biochemical assays for small molecule inhibitors and activators of specific functions. In addition, the facility provides services to aid investigators in chem-informatics, lead expansion, and analysis of cytotoxic effects of candidate molecules. Specifically, the SMSF has expertise in queries of data bases such as PubChem, a large database that is maintained by the NCBI and that can be searched by substance, compound, bioactivity, or structure, and that can be used to identify series of known compounds with bioactivity or structures similar to those of candidate compounds. This resource may allow rapid studies of the relationship of structure and function in candidate compounds before resorting to medicinal chemistry. The toxicity screening function of the SMSF is based on the in vitro anticancer drug screen system of the Cancer Treatment & Diagnosis division of the NCI. It includes a full library containing 60 human tumor cell lines representing various tumor types. The SMSF has developed their own pre-screening system that contains ten tumor cell lines used for initial testing of most compounds.

293T human embryo kidney cells (HEK-293T cells) or COS 7 cells are transfected with the mammalian two-hybrid plasmids, followed by a stepwise screening of the SMSF libraries and validation of the ability of compounds to induce apoptosis in human mast cell and melanoma lines. pFR-LUC is used in the robotic systems of the SMSF as an alternate to the CAT reporter plasmid. Like the CAT reporter plasmid, pFRLuc contains multiple GAL4 promoter sequences but they control a Luciferase gene cDNA rather than the CAT gene.

Candidate compounds are further tested for their ability to induce apoptosis in MAGE positive human melanoma cell lines in vitro. Results showed that the large scale calcium phosphate technique results in at least 95% transfection efficiency with HEK-293T cells, and are sufficient for screening in the SMSF. Many standard techniques are also used for establishing stably transfected cell lines.

NCI Libraries (NCI01, NCI02, and NCI03, described in more detail in the following section) are relatively small libraries, and are first screened before advancing to the larger and more complex libraries. For instance, initial screening allow the determination of basic parameters such as the number of cells/well (usually between 3,000 and 10,000) necessary for detection with the reporter system, general toxicity statistics for comparison with those of previous users of the facility, the range of concentrations of compounds to be used, and whether there is a need to use the 94 well plate format or the more efficient 384 well format. The Known Bioactive Library, a library of intermediate complexity composed of 4160 molecules with known biologically activity. Finally, two highly complex libraries, the Chembridge and ChemDiv Libraries are screened.

Promising molecular candidates are screened for general toxicity and LC50 dosages determined in the SMSF. Promising compounds are further screened for their ability to induce apoptosis in HMC1.1 and HMC1.2 cells and to kill the Hs-294T and A375 human melanoma cell lines. NIH3T3 cells will serve as additional negative controls.

The NCI Libraries: NCI01, NCI02, and NCI03: The NCI01 library consists of 1990 compounds selected from the NCI open collection of 140,000 compounds. The compounds were selected based upon availability and drug-likeness. Also included in the NCI01 library is a challenge set of 57 compounds of novel structural types which have shown unusual patterns of cell line sensitivity and resistance. The NCI02 library consists of 879 compounds which have represent a broad range of structural diversity and patterns of growth inhibition in the NCI 60 cell line screen. The NCI03 library consists of 235 natural products that were selected from the NCI open repository on the basis of structural diversity and availability of compound.

Known Bioactive Library (KBA01): this library consists of 3 commercially available collections totaling 4,160 compounds. KBA01 consists of 880 high purity compounds of known safety and bioavailability in humans of which over 85% are marketed drugs from Prestwick Chemical. The Prestwick compounds cover several therapeutic areas including neuropsychiatry, cardiology, immunology, inflammation, analgesia, etc. Also included in the KBA01 library are 2000 diverse FDA approved drugs and natural products from the Spectrum Chemical Collection (Microsource Discovery Systems, Inc.) and 1280 compounds from the Sigma LOPAC collection (Library of Pharmacologically Active compounds). These compounds represent marketed drugs, failed development candidates and “gold standards” that have well-characterized pharmacologic activities. These compounds are the results of lead optimization efforts and have been rationally designed by structure-activity relationship studies.

Chembridge Library: The SMSF possesses a 16,000 compound library from the Chembridge DIVERSet. These are a collection of “universally” diverse, pre-designed drug-like small molecules. The compounds have been rationally selected based on 3D pharmacophore analysis to cover the broadest part of biologically relevant pharmacophore diversity space.

ChemDiv Library: This library is a 20,000 member compound library from Chemical Diversity Labs, inc. It was chosen to complement the Chembridge library. Chemical Diversity Labs examined the current collection of compounds in the SMSF and ran heterocyclic clustering and matrix maximization algorithms of Chemsoft™ to fill voids in the SMSF chemistry space while maintaining maximal diversity of the entire set. All of these collections have been combined and reformatted for screening in 13-384 well plates and are available for screening in 384-well or 96-well plates.

As in any screening schemes, false positives and false negatives have to be removed. Potential false positives include compounds that are generally cytotoxic, that are general inducers of apoptosis, or that generally suppress transcription. These compounds would decrease the Luciferase signal and therefore be read as positive in our assays. Most compounds with general cytotoxicity have already been identified in the SMSF and can be confirmed in the toxicity screen or by simply incubating the compound with the HEK-293T cells or NIH3T3 cells, and checking viability. Likewise, general suppressors of transcription have already been identified, and additional similar compounds could be identified and confirmed by their failure to induce apoptosis in the mast cell and melanoma cell lines. General inducers of apoptosis will also induce apoptosis in NIH3T3 cells and other control cell lines.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof. All references cited hereinabove and/or listed below are hereby expressly incorporated by reference.

1. A method for inhibiting the growth or proliferation, or inducing apoptosis, of a cell that expresses a MAGE gene, the method comprising inhibiting the expression of the MAGE gene or inhibiting the function of a polypeptide encoded by the MAGE gene in the cell.

2. A method according to claim 1, wherein the method comprising administering to the cell a polynucleic acid molecule that specifically inhibits the expression of the MAGE gene, or a polypeptide that specifically inhibits the function of a protein encoded by the MAGE gene in the cell.

3. A method according to claim 2, wherein the polynucleic acid molecule is a short interfering RNA molecule, or an antisense nucleic acid molecule that is specific against the MAGE gene, or a nucleic acid molecule that forms a triplex with the MAGE gene, thereby inhibiting the expression of the MAGE gene.

4. A method according to claim 2, wherein the polypeptide is an antibody against the MAGE gene or MAGE gene product.

5. A method according to claim 4, wherein the antibody is a monoclonal antibody, or an active fragment thereof.

6. A method according to claim 5, wherein the antibody is a humanized antibody.

7. A method according to claim 5, wherein the antibody is a human antibody.

8. A method according to claim 1, wherein the MAGE gene is a Type I MAGE gene.

9. A method according to claim 8, wherein the Type I MAGE gene is MAGE-A, MAGE-B, or MAGE-C.

10. A method according to claim 8, wherein the Type I MAGE gene is MAGE-A1, A3, A5, A6, A8, A9, A10, A11 or A12, and the MAGE-B gene is MAGE-B1, B2, B3 or B4.

11. A method according to claim 1, wherein the MAGE gene is a Type II MAGE gene.

12. A method according to claim 11, wherein the Type II MAGE genes is Necdin, MAGE-D, MAGE-E (E1), MAGE-F, MAGE-G, or MAGE-H.

13. A method according to claim 1, wherein the cell is a cancerous or malignant or neoplastic cell.

14. A method according to claim 13, wherein the cancer, tumor, or cellular proliferation is selected from the group consisting of melanomas, lymphoma, T-cell leukemia, non-small cell lung carcinoma, hepatic carcinoma, gastric cancer, esophagus carcinomas, colorectal carcinomas, pancreatic endocrine neoplasms, ovarian neoplasms, cervical cancer, salivary glands carcinoma, head and neck squamous cell carcinomas, spermatogonia, proliferating or non-proliferating testes cells, spermatocytic seminoma, sporadic medullary thyroid carcinoma, osteosarcomas, childhood astrocytomas, bladder cancer, cells from inflamed joints in juvenile rheumatoid arthritis or other harmful inflammatory condition, glioma, neuroblastoma tumors, and cancers related to malignant mast cells.

15. A method for treating a cancer, tumor, or cellular proliferation in a mammal, wherein the cancer or tumor comprises a cell that expresses a MAGE gene, the method comprising inhibiting the expression of the MAGE gene or inhibiting the function of a polypeptide encoded by the MAGE gene of the cell in the mammal.

16. A method according to claim 15, wherein the method comprising administering to the cell a polynucleic acid molecule that specifically inhibits the expression of the MAGE gene, or a polypeptide that specifically inhibits the function of a protein encoded by the MAGE gene in the cell.

17. A method according to claim 16, wherein the polynucleic acid molecule is a short interfering RNA molecule, or an antisense nucleic acid molecule that is specific against the MAGE gene, or a nucleic acid molecule that forms a triplex with the MAGE gene, thereby inhibiting the expression of the MAGE gene.

18. A method according to claim 16, wherein the polypeptide is an antibody against the polypeptide encoded by a MAGE gene.

19. A method according to claim 1, wherein the cell is a cell in a testes.

20. A method according to claim 19, wherein the cell is a male germ cell.

21. A pharmaceutical composition for treating tumor cell formation, tumor cell growth, or a testes cell comprising an antagonist to MAGE and a pharmaceutically acceptable excipient.

22. A method for screening for a substance that inhibits MAGE gene expression, the method comprising: (1) providing a candidate substance to be tested; (2) applying said candidate substance to a cell expressing a MAGE gene or MAGE gene construct, (3) detecting the level of expression of the MAGE gene or MAGE gene construct in the presence of the candidate substance, and (4) determining a level of expression of the MAGE in the absence of the candidate substance, wherein a substance that decreases the level of expression of the MAGE gene is selected.
